(19) United States
(12) Patent Application Publication
(10) Pub. No.: US 2025/0090047 A1
(43) Pub. Date: Mar. 20, 2025

(54) 2-D POSE ESTIMATION-BASED AUTOMATED PASSIVE RANGE OF MOTION MEASUREMENT TOOL

(71) Applicant: My Medical HUB Corporation, Safety Harbor, FL (US)

(72) Inventors: Walter M Groteke, Safety Harbor, FL (US); Barry Fitch, Augusta, SC (US); Md. Al Mehedi Hasan, Rajshahi (BD); Rifaet Ullah, Rangpur (BD)

(21) Appl. No.: 18/885,093

(22) Filed: Sep. 13, 2024

Related U.S. Application Data
(60) Provisional application No. 63/582,564, filed on Sep. 14, 2023.

(51) Int. Cl. A61B 5/11; A61B 5/00

(52) U.S. Cl. CPC A61B 5/1121; A61B 5/1128; A61B 5/0077

(57) ABSTRACT

A method of measuring a patient's range of motion being operable on a computer system having a database, and a device comprising a camera, the method having the steps of recording a video of the patient as they proceed through a stretching procedure, the video having frames of the patient in rest and fully stretched poses; capturing the frames of the patient in the rest and fully stretched poses; identifying key-points within the frames; tracing the range of movement for the key-points in the frames; calculating a range of motion angle by comparing key-point positions between the frames of the rest and fully stretched poses; recording the range of motion angle; and reporting the range of motion angle to the patient or a clinician. The patient may be assisted through the stretching procedure by a clinician, wherein the method is configured to ignore the presence and movement of the clinician.

### CROSS-REFERENCE TO RELATED APPLICATIONS

This application claims the benefit of U.S. Provisional application No. 63/582,564, filed Sep. 14, 2023, which is hereby incorporated by reference, to the extent that it is not conflicting with the present application.

### BACKGROUND OF INVENTION

### 1. Field of the Invention

The invention relates generally to systems and methods for analyzing patients and specifically to systems and methods for pose estimation for automatically measuring a patient's passive range of motion.

### 2. Description of the Related Art

In assessing a patient's overall health, physicians, clinicians or other medical providers will often measure different aspects of a patient's physical capabilities, including the patient's range of motion. One type of range of motion measurement that may be collected is a patient's “passive” range of motion, wherein clinician, physician or other trained personnel physically move the patient through a corresponding range of motion exercise by posing the patient in the correct positions. The current methods for attempting to accurately measure a patient's passive range of motion (“PROM”) may require clinicians to simultaneously brace, grip, and move various regions of the patient's body while obtaining measurements using manual goniometer techniques. The simultaneous manipulation of the patient and a manual goniometer or other measurement device cannot occur simultaneously without being prone to errors, inaccuracies, and inconsistencies. Furthermore, while using the current manual goniometer methodology, the provider/clinician has to either stop to record the measurement or engage an assistant to scribe or enter the data, thus taking additional time or requiring additional personnel.

Therefore, there is a need to solve the problems described above by proving a device and method for automatically measuring passive range of motion values for a patient through the utilization of two dimensional pose estimation techniques.

The aspects or the problems and the associated solutions presented in this section could be or could have been pursued; they are not necessarily approaches that have been previously conceived or pursued. Therefore, unless otherwise indicated, it should not be assumed that any of the approaches presented in this section qualify as prior art merely by virtue of their presence in this section of the application.

### BRIEF INVENTION SUMMARY

This Summary is provided to introduce a selection of concepts in a simplified form that are further described below in the Detailed Description.

In an aspect, a method of measuring a patient's range of motion is provided, the method of measuring a patient's range of motion being operable on a computer system having a database, and a device comprising a camera, the method comprising: recording a video of the patient as they proceed through a stretching procedure, the video comprising: a frame of the patient in a rest pose and a frame of the patient in a fully stretched pose; capturing the frames of the patient in a rest pose and the fully stretched pose; identifying key-points within the frames; tracing the range of movement for the key-points in the frames; calculating a range of motion angle by comparing key-point positions between the frames; recording the range of motion angle; and reporting the range of motion angle to the patient or a clinician. Thus, an advantage is that a patient's passive range of motion may be accurately measured without the need for auxiliary sensors or manual measurement devices, such as a manual goniometer. Another advantage is that the pictures/frames of the patient in the rest pose and the fully stretched pose, and thus the resultant PROM angles, may be captured automatically by the computer system, thus allowing the clinician to suitably assist the patient through the stretching procedure without requiring additional personnel or additional time to record the results. Another advantage is that the resultant PROM values or other calculations may be provided to the patient or clinician as part of a comprehensive report, wherein an overview of the patient's health may be provided to interested parties.

The above aspects or examples and advantages, as well as other aspects or examples and advantages, will become apparent from the ensuing description and accompanying drawings.

### BRIEF DESCRIPTION OF THE DRAWINGS

For exemplification purposes, and not for limitation purposes, aspects, embodiments or examples of the invention are illustrated in the figures of the accompanying drawings, in which:

FIGS. 1A-1C illustrate the procedure for a patient and clinician performing a left shoulder abduction passive range of motion measurement test, according to an aspect.

FIG. 1D illustrates an image of a patient in a fully stretched pose for the left shoulder abduction passive range of motion test, according to an aspect.

FIGS. 2A-2C illustrate the procedure for a patient and clinician performing a right shoulder flexion passive range of motion measurement test, according to an aspect.

FIG. 2D illustrates an image of a patient in a fully stretched pose for the right shoulder flexion passive range of motion test, according to an aspect.

FIGS. 3A-3C illustrate the procedure for a patient and clinician performing a left shoulder internal rotation passive range of motion measurement test, according to an aspect.

FIG. 3D illustrates an image of a patient in a fully stretched pose for the left shoulder internal rotation passive range of motion test, according to an aspect.

FIGS. 4A-4C illustrate the procedure for a patient and clinician performing a thoracolumbar flexion passive range of motion measurement test, according to an aspect.

FIG. 4D illustrates an image of a patient in a fully stretched pose for the thoracolumbar flexion passive range of motion test, according to an aspect.

FIGS. 5A-5C illustrate the procedure for a patient and clinician performing a thoracolumbar extension passive range of motion measurement test, according to an aspect.

FIG. 5D illustrates an image of a patient in a fully stretched pose for the thoracolumbar extension passive range of motion test, according to an aspect.

FIGS. 6A-6C illustrate the procedure for a patient and clinician performing a thoracolumbar right lateral flexion passive range of motion measurement test, according to an aspect.

FIG. 6D illustrates an image of a patient in a fully stretched pose for the thoracolumbar right lateral flexion passive range of motion test, according to an aspect.

FIGS. 7A-7C illustrate the procedure for a patient and clinician performing a right thoracolumbar rotation passive range of motion measurement test, according to an aspect.

FIG. 7D illustrates an image of a patient in a fully stretched pose for the right thoracolumbar rotation passive range of motion test, according to an aspect.

FIGS. 8A-8C illustrate the procedure for a patient and clinician performing a right knee flexion passive range of motion measurement test, according to an aspect.

FIG. 8D illustrates an image of a patient in a fully stretched pose for the right knee flexion passive range of motion test, according to an aspect.

### DETAILED DESCRIPTION

What follows is a description of various aspects, embodiments and/or examples in which the invention may be practiced. Reference will be made to the attached drawings, and the information included in the drawings is part of this detailed description. The aspects, embodiments and/or examples described herein are presented for exemplification purposes, and not for limitation purposes. It should be understood that structural and/or logical modifications could be made by someone of ordinary skills in the art without departing from the scope of the invention.

For the following description, it can be assumed that most correspondingly labeled elements across the figures (e.g., 101, 201, etc.) possess the same characteristics and are subject to the same structure and function. If there is a difference between correspondingly labeled elements that is not pointed out, and this difference results in a non-corresponding structure or function of an element for a particular embodiment, example or aspect, then the conflicting description given for that particular embodiment, example or aspect shall govern.

It should be understood that, for clarity of the drawings and of the specification, some or all details about some structural components or steps that are known in the art are not shown or described if they are not necessary for the invention to be understood by one of ordinary skills in the art.

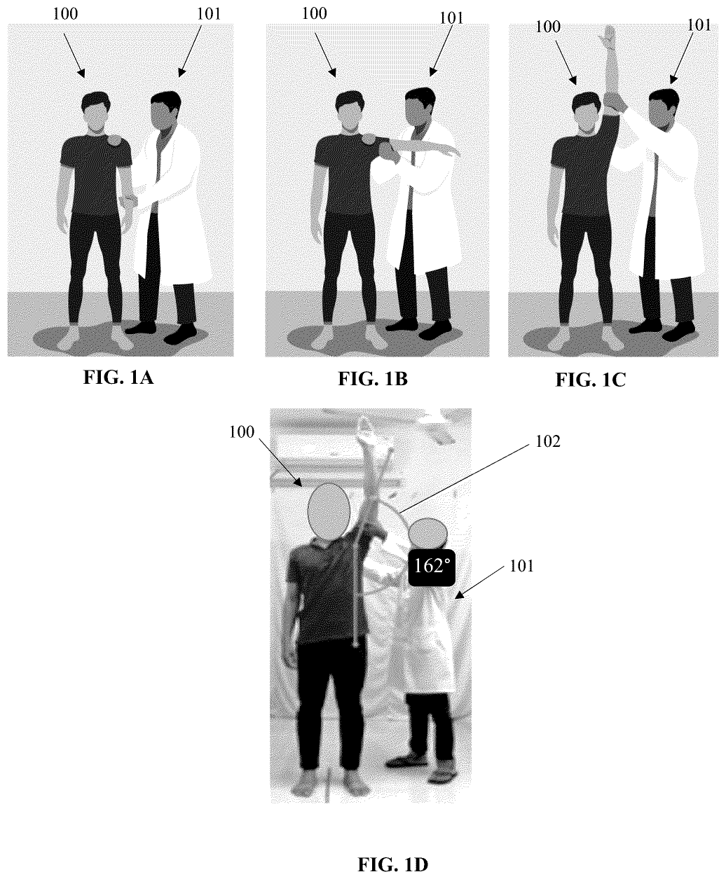

FIGS. 1A-1C illustrate the procedure for a patient 100 and clinician 101 performing a left shoulder abduction passive range of motion measurement test, according to an aspect. FIG. 1D illustrates an image of a patient 100 in a fully stretched pose for the left shoulder abduction passive range of motion test, according to an aspect. In order for a patient 100 to have their passive range of motion measured for a particular stretching procedure, the patient 100 may be assisted by an on-site clinician 101, and may utilize a specific tool within a “MyMedicalHub” (“MMH”) computer system. MyMedicalHUB has developed a standardized, automated tool that uses 2-D (“two dimensional”) pose estimation to generate PROM data in an unbiased manner. As a result of these herein disclosed stretching procedures being executed by the patient 100 with the physical guidance of a clinician 101, the collected clinician assisted range of motion data may be referred to as PROM measurements/values.

It should be understood that passive range of motion is the ROM (“range of motion”) that is achieved when an outside force (e.g., a clinician, therapist, CPM machine, etc.) exclusively causes movement of a patient's joint. Said passive range of motion may be the maximum range of motion that a joint can move without injury. Passive range of motion procedures may be performed in instances in which the patient 100 is unable or not permitted to move a corresponding body part linked to measuring their range of motion for a particular region.

The disclosed MyMedicalHUB computer system is a cloud-based technology that automates and captures data that has been traditionally generated manually by clinicians (“providers”) 101. Patients 100 are invited by a clinician 101 to log-in to MyMedicalHUB via a secure link, register, and complete a comprehensive online physical assessment. The comprehensive online physical assessment may include subjective intake questions consistent with in-office evaluations and management examinations, such as activities of daily living (ADLs), personal and family medical history, chief complaint(s), and, in some cases, subjective survey tools, such as an Oswestry Disability Index or STEADI Fall Risk Assessment Questionnaire. The MMH system may include a database in which the collected patient data, including health information, as well as recorded videos, relevant frames/pictures from the recorded videos, and relevant angle measurements are stored for later use. It should be understood that the term “frame” and “picture” may be utilized interchangeably herein, wherein the frames/pictures correspond to the individual images that make up a recorded video. Once the subjective intake questions are complete, the patient 100 may be led through a series of provocative movements that measure and record range of motion using a computer, tablet, cell phone, smart device or other suitable electronic device paired with a video camera (“camera”) configured to take two dimensional videos, as opposed to a traditional goniometers and other sensor or devices related to signaling technology. These provocative movements (“stretching procedures,” “PROM tests”) may be “performed” with both a patient 100 and clinician/provider 101 in the same instance, wherein the clinician/provider 101 manipulates the body of the patient 100 to perform these stretching procedures. These PROM measurements may be compared to normative ranges as well as used to track objective data on a longitudinal basis. Following each movement, the MMH system also asks specific questions that encompass symptomatic criteria outlined in published, peer reviewed clinical studies and evidenced-based medicine protocols.

For PROM tests, patients 100 may position themselves in front of the camera based on the movement that is being measured. Clinicians 101 may position themselves using predefined protocols that do not interfere with the camera's ability to capture the patient's movements in an unencumbered way, i.e., to the left, right, or behind the patient. As clinicians 101 “assist” patients 100 in performing these movements, thus allowing them to proceed through corresponding stretching procedures, the MMH AI technology may capture the movement of the patient, superimpose virtual markers on specific key-points on the patient, trace the range of movement, calculate the corresponding passive range of motion angle at the maximum point of movement (e.g. the fully stretched position), measure it in degrees, and feed the collected information into a comprehensive report. These movements/stretching procedures may be captured by a cell phone, tablet, computer, smart device or other device having a camera and an internet connection, wherein both the patient 100 and the clinician 101 may be seen and clearly distinguished from each other in each frame of the captured video, live feed, etc. As is understood, the disclosed process of tracking key-points overlayed on the captured frames of the recorded video in order to determine the passive range of motion of a patient 100 may be done without the use of any additional sensors or manual tools. As a result of this, the utilization of said additional devices may be avoided, thus preventing the excess variations associated with said devices.

In an embodiment, the disclosed method of two dimensional pose estimation may be utilized in order to measure a patient's left shoulder abduction PROM. For this left shoulder abduction PROM test, the patient 100 may stand squarely facing the camera with their feet shoulder width apart. The patient 100 may have their palms facing their body with their thumbs facing towards the camera, as seen in FIG. 1A. This pose of FIG. 1A may be referred to as the rest pose for the corresponding PROM test, wherein the patient 100 has not begun stretching. The MMH system may be configured to utilize the corresponding camera to record a video of the patient 100 (and clinician 101) while the patient 100 is in the rest pose of FIG. 1A, to compare to later portions of the video. This trend of recording a video of the patient 100 proceeding through a stretching procedure, from the rest pose to the fully stretched pose may be repeated for each PROM test disclosed herein. At this point, the patient 100 may allow the clinician 101 to move said patient's arm through the designated range to assess the patient's left shoulder abduction PROM.

The clinician 101 may follow a complementary procedure in order to properly manipulate the patient 100 to determine the patient's left shoulder abduction PROM. In order to allow the camera to record the required video without being blocked, the clinician 101 may stand to the left rear of the patient 100 during the procedure, as shown in FIG. 1A. From this position, the clinician 101 may place their right hand on the lateral border of the patient's left shoulder for stabilization and place their left hand on the patient's left humerus for movement of the patient's left arm. The clinician 101 may then move the patient's left arm through the range of abduction to tolerance, to a maximum referred to as the patient's fully stretched pose, as seen in FIG. 1C. While the patient is in this fully stretched pose of FIG. 1C, the clinician 101 may allow the MMH system to automatically record video footage and take measurements. The measurements captured by the MMH system may be automatically uploaded into the patient's electronic medical record for easy access by the patient and their care team (physicians, clinicians, etc.) It should be understood that the MMH system may be configured to automatically record a video of the patient 100 while said patient is in the fully stretched pose of FIG. 1C, or the clinician 101 may have some hands free mechanism (e.g., voice commands) to indicate to the MMH system when the patient 100 is in the fully stretched pose. As can be seen in the embodiment of FIG. 1D, the patient 100 may have a PROM angle (“passive range of motion angle”, “PROM value”, “PROM measurement”) 102 of about 162° for the left shoulder abduction PROM test.

As disclosed hereinabove, the MMH system may utilize a standardized, automated method that uses two dimensional pose estimation to gather/generate accurate passive range of motion data for the patient 100, while both the patient 100 and the clinician 101 are included within each frame of the recorded video. With both the patient and clinician within the same frame (e.g., both the patient and clinician are within the field of vision of the camera), the MMH system is trained to first distinguish the clinician by virtue of his/her positioning, then lock-in focus on the patient throughout each movement. The MMH system may be configured to only identify key-points on the patient, while ignoring any potential points on the clinician. In an embodiment, clinicians are provided with specific instructions relating to both body and hand positioning. The MMH system may be configured to distinguish between patient 100 and clinician 101, lock-in on the patient 101, superimpose “marker-less” sensors, track, and measure passive range of motion for various joints within the patient's body. As such, the MMH system may be configured to facilitate more efficient time management by utilizing its automated AI-based PROM measurement method to capture and record the necessary information to provide accurate PROM values without the need for additional personnel or additional time to self-record. It should be understood that the above described technique of identifying and tracing key-points from the relevant frames of the recorded video may be utilized in various different PROM measurement tests, as will be described in greater detail hereinbelow.

In general, the overall process of measuring a patient's range of motion utilizing the disclosed two-dimensional pose estimation method may be summarized in a few steps. First the MMH system may utilize a corresponding camera to record a video of the patient as they are taken through a stretching procedure. As the camera records the entirety of the stretching video, said video may comprise at least a frame of the patient in a rest pose and a frame of the patient in a fully stretched pose. From here, the MMH system may capture the frames of the patient in a rest pose and the fully stretched pose for analysis. With these relevant frames captured, the MMH system may identify key-points within the frames and trace the range of movement for the key-points in the frames. Next, the MMH system may thusly calculate a corresponding range of motion angle for the stretching procedure by comparing clinician assisted key-point positions at the rest pose and fully stretched pose frames. Finally, this PROM value may be recorded and reported to the patient, clinician, or any other party of interest. This generalized process may be utilized for each PROM measurement test disclosed herein, wherein the patient may allow the clinician to move the patient's body accordingly to assume the necessary poses to acquire the PROM measurement.

As disclosed hereinabove, several key-points may be identified and tracked as a patient moves through a particular procedure in order to facilitate measurement of a patient's passive range of motion as disclosed hereinabove. In an embodiment, for the left shoulder abduction passive range of motion measurement test shown in FIG. 1A-1D, a first key-point may be the anterior superior iliac spine (ASIS) of the patient's left hip, a second key-point may be the glenohumeral joint of the patient's left shoulder and a third key-point may be the medial epicondyle of the patient's left elbow. As should be understood, “key-points” may only be identified on the patient, despite having a clinician in frame while recording the patient, as the positioning of the clinician's body may not be relevant to measuring a patient's PROM value.

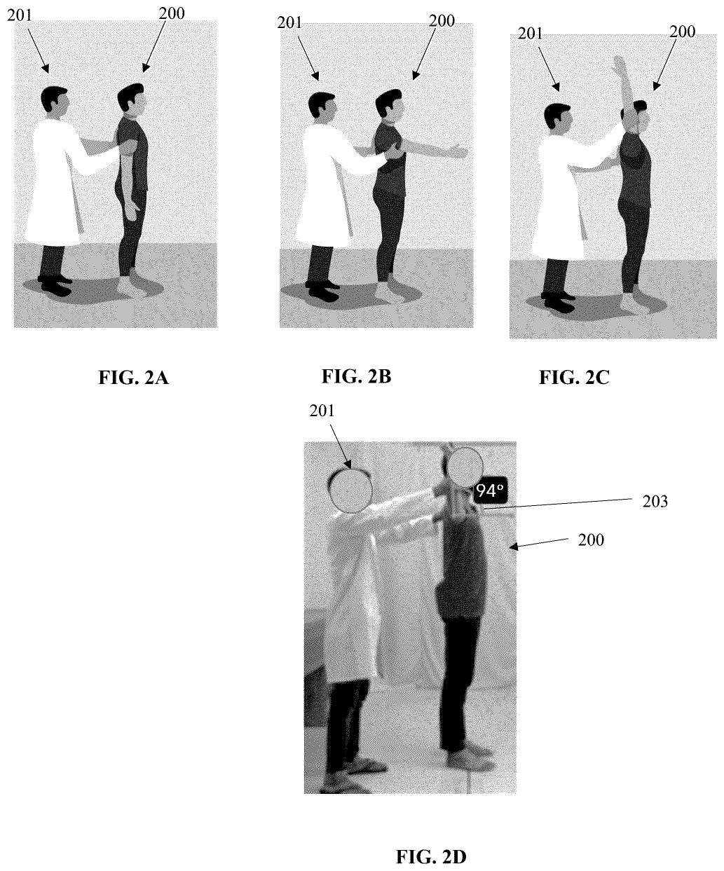

FIGS. 2A-2C illustrate the procedure for a patient 200 and clinician 201 performing a right shoulder flexion passive range of motion measurement test, according to an aspect. FIG. 2D illustrates an image of a patient 200 in a fully stretched pose for the right shoulder flexion passive range of motion test, according to an aspect. Similarly to the left shoulder abduction PROM test disclosed hereinabove in FIG. 1A-1D, the right shoulder flexion PROM test of FIG. 2A-2D may track particular portions of a patient's body in order to assess the patient's range of motion for the right shoulder flexion stretching procedure.

In an embodiment, the disclosed method of two dimensional pose estimation may be utilized in order to measure a patient's right shoulder flexion PROM. For this right shoulder flexion PROM test, the patient 200 may stand with their right side squarely facing the camera and their feet shoulder width apart with their arms at their sides. While in this position, the back of the patient's hands should be facing the camera with their thumbs pointed forward. This pose may be identified as the rest post for the right shoulder flexion PROM test, as seen in FIG. 2A. At this stage, the patient 200 may allow the clinician 201 to move the patient's right arm through their full range of motion.

The procedure utilized to perform the designated right shoulder flexion PROM test may involve moving a corresponding limb of the patient 200, in this case the patient's right arm. In this PROM test, the clinician 201 may stand at the right rear of the patient 200, as seen in FIG. 2A. The clinician 201 may then place their left hand on the patient's right scapula for stabilization while placing their right hand on the patient's right mid-humerus while being mindful not to block the camera's view of the patient's elbow or lateral shoulder. At this point, the clinician 201 may move the patient 200 through the full range to their tolerance for their right arm. Once at the fully stretched pose for this PROM measurement, as shown in FIG. 2C, the clinician 201 may allow the MMH system to take corresponding PROM measurements, as described hereinabove. Again, the MMH system may be configured such that a frame of the patient 200 in the rest pose, as seen in FIG. 2A, and a frame of the patient 200 in the fully stretched pose of FIG. 2C are captured as part of the recorded video for analysis. It should also be understood that intermediate poses, such as the intermediate pose of FIG. 2B may also be captured in a corresponding frame, for applications in which having said intermediate pose frame may prove useful.

In an embodiment, tracking the entire movement of the stretching procedure for a PROM test from the rest pose of FIG. 2A, through the intermediate pose of FIG. 2B, and to the fully stretched pose of FIG. 2C may also facilitate the identification of abnormalities and/or limitations in the patients PROM. As such, while the intermediate poses may not be utilized in ROM measurements, they may provide other insights relevant to patient health. Furthermore, frames of/data from said poses, including intermediate poses, may be made available to the patient and their care team (physician, doctor, clinicians, etc.) in instances in which it is helpful to do so.

Regardless of the specific range of motion test being performed, the MMH system may be configured to accurately track corresponding portions of a patient's body between a rest pose, such as the rest pose of FIG. 2A, and a fully stretched pose, such as fully stretched pose of FIG. 2C. As with the left shoulder abduction PROM measurement test of FIG. 1A-1D, the MMH system may be configured to capture the movement between the rest pose and the fully stretched pose, superimpose virtual markers on specific key-points, trace the range of movement, calculate the angle at the maximum point of movement, measure it in degrees, and feed the information into a comprehensive report. Again, it should be understood that the MMH system may be configured to repeat this above process of utilizing two dimensional pose estimation to calculate a patient's passive range of motion for each pose stretching procedure/PROM test described herein, as well as any other stretching procedure that may be anticipated in which the key-points required to measure a patient's PROM may be observed and tracked for comparing corresponding angles between the rest and fully stretched poses. In the present embodiment of FIG. 2D, the patient's PROM angle 203 for the described right shoulder flexion PROM test may be about 94°. Additional PROM measurement procedures for different body regions will be outlined hereinbelow, all of which may be conducted through utilization of the disclosed MMH system, and utilize two-dimensional pose tracking, as disclosed hereinabove.

As is understood, several key-points may be identified and tracked in order to facilitate the measurement of the patient's passive range of motion. In an embodiment, for the disclosed right shoulder flexion passive range of motion measurement test of FIG. 2A-2D, a first key-point may be a point parallel to the floor from the olecranon process of the patient's right elbow, a second key-point may be the olecranon process of the patient's right elbow and a third key-point may be the ulnar styloid process of the patient's right wrist.

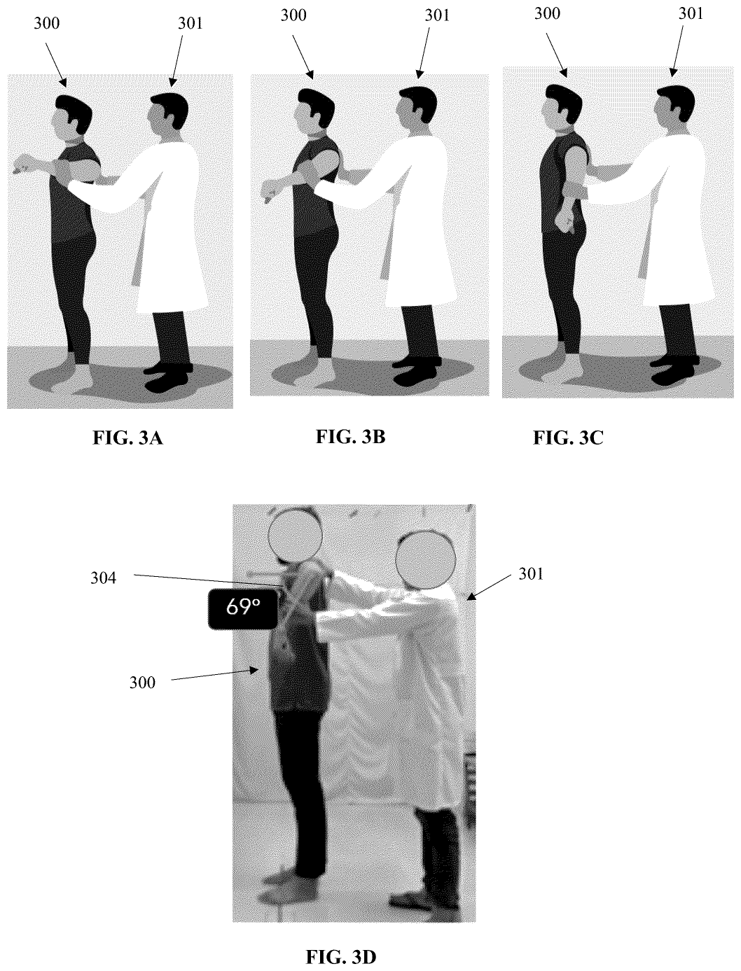

FIGS. 3A-3C illustrate the procedure for a patient 300 and clinician 301 performing a left shoulder internal rotation passive range of motion measurement test, according to an aspect. FIG. 3D illustrates an image of a patient 300 in a fully stretched pose for the left shoulder internal rotation passive range of motion test, according to an aspect. Similarly to the left shoulder abduction PROM test disclosed hereinabove in FIG. 1A-1D, the left shoulder internal rotation PROM test of FIG. 3A-3D may track particular portions of a patient's body in order to assess the patient's range of motion for the left shoulder internal rotation stretching procedure outlined hereinbelow.

To begin the left shoulder internal rotation PROM test, the patient 300 may stand squarely with their left side facing the camera, and their arms relaxed to their sides, wherein said pose may be described as the rest pose for the left shoulder internal rotation PROM test, as shown in FIG. 3A. The patient 300 may keep their arms relaxed while allowing their clinician 301 to move their left arm through the necessary motion to assess their left shoulder internal rotation PROM. As disclosed hereinabove, the patient 300 may typically remain still while performing passive range of motion exercises, whereas the clinician 301 may be responsible for actually moving the corresponding portion of the patient 300 to perform the corresponding stretching procedure.

For the clinician side of left shoulder internal rotation PROM test, the clinician 301 may stand to the right rear of the patient 300, again making sure to not obstruct the camera's view of the patient 300, or any of the patient's corresponding key-points. The clinician 301 may place their right hand on the posterior aspect of the patient's left humerus for stabilization, wherein the clinician may place their left hand 2-3 inches above the patient's left wrist. From here, the clinician 301 may move the patient 300 through left shoulder internal rotation to tolerance. Again, once the patient 300 is at the fully stretched pose, as seen in FIG. 3C, for the passive range of motion test (e.g., the patient is at tolerance), the clinician 301 may allow the MMH system to capture a corresponding image to compare to the rest pose of FIG. 3A to determine the patient's left shoulder internal rotation PROM. Again, whether or not the intermediate pose of FIG. 3B is captured or utilized may depend on if the intermediate pose has a diagnostic use, or is otherwise desirable to record. As seen in the fully stretched pose of FIG. 3D, the patient 300 may have a left shoulder internal rotation PROM angle 304 of about 69°.

Again, several key-points may be identified and tracked as the patient moves through a particular PROM test in order to facilitate the measurement of a patient's passive range of motion. In an embodiment, for the left shoulder internal rotation passive range of motion measurement test of FIG. 3A-3D, a first key-point may be a point parallel to the floor from the olecranon process of the patient's left elbow, a second key-point may be the olecranon process of the patient's left elbow and a third key-point may be the ulnar styloid process of the patient's left wrist.

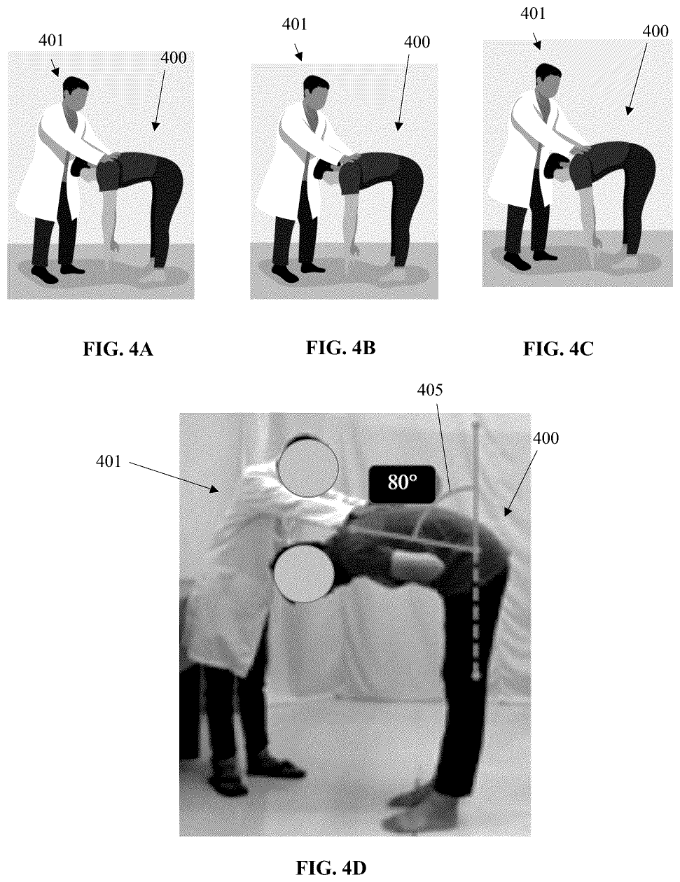

FIGS. 4A-4C illustrate the procedure for a patient 400 and clinician 401 performing a thoracolumbar flexion passive range of motion measurement test, according to an aspect. FIG. 4D illustrates an image of a patient 400 in a fully stretched pose for the thoracolumbar flexion passive range of motion test, according to an aspect.

For the patient side of the thoracolumbar flexion PROM test, the patient 400 may stand while lined up squarely with their left side facing the camera, and may allow their arms to relax. Once again, this pose may be referred to as the rest pose, as seen in FIG. 4A. The patient 400 may keep their knees locked while allowing the clinician to pull them forward through their thoracolumbar flexion range of motion. The patient 400 may then hold this fully stretched pose until the measurement is taken (e.g., until the video of the patient 400 in the fully stretched pose of FIG. 4C is recorded).

For the clinician side of thoracolumbar extension PROM test, the clinician 401 may stand in front of and “caddy corner” to the patient 400 (diagonally to, in front and to the right of the patient 400), as to not block the camera's view of the patient 400. The clinician 401 may place both of their hands on the patient's upper back at approximately T4 (the patient's fourth thoracic vertebra) and provide gentle downward pressure to tolerance. Upon reaching the fully stretched pose of FIG. 4C, at which the patient 400 is at tolerance for the thoracolumbar flexion PROM test, the clinician 401 may allow the MMH system to take the recording/measurement, as disclosed hereinabove. In an embodiment, the clinician 401 may initiate the automated capture of movement data/video recording by the MMH system by pressing a “start” arrow button on a corresponding device for the specific movement. For this PROM test, the clinician 401 must take care to avoid obstructing the camera's view of the knee, hip and shoulder of the patient 400, all of which may correspond to key-points identified and tracked for the corresponding PROM measurements. The clinician 401 should also be careful not to stand behind the patient's head as best as possible. By tracking the key-points on the patient 400 between the rest pose of FIG. 4A and the fully stretched pose of FIG. 4C, the patient's PROM angle 405 for their thoracolumbar flexion may be determined to be about 80°, as seen in the fully stretched pose of FIG. 4D.

In order to provide clinicians with a solid insight into patient health, clinicians may also utilize the MMH system to examine previously recorded videos, captured pictures/frames and annotations, accordingly. Furthermore, clinicians may also be given the option to manually make edits to the measured values if they believe the PROM measurement for a test are inaccurate. By providing a means of correcting any potential issues that may occur during data collections, the MMH system may provide clinicians with a versatile tool that they may utilize in conjunction with their expertise to acquire highly accurate data and thus provide patients with accurate and helpful diagnoses.

As disclosed hereinabove, each passive range of motion test may have a plurality of key-points that may be tracked in order to calculate a patient's range of motion. In an embodiment, for the thoracolumbar flexion passive range of motion measurement test of FIG. 4A-4D, a first key-point may be a point perpendicular to the floor from the femoral head of the patient's left hip, a second key-point may be the femoral head of the patient's left hip and a third key-point may be the superior lateral border of the patient's left scapula.

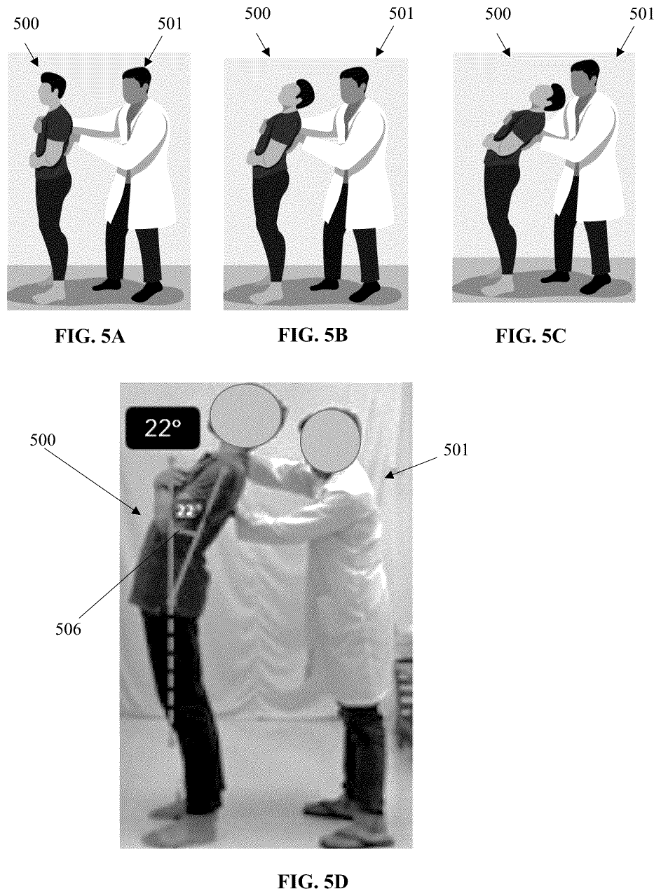

FIGS. 5A-5C illustrate the procedure for a patient 500 and clinician 501 performing a thoracolumbar extension passive range of motion measurement test, according to an aspect. FIG. 5D illustrates an image of a patient 500 in a fully stretched pose for the thoracolumbar extension passive range of motion test, according to an aspect.

For the patient side of the thoracolumbar extension PROM test, the patient 500 may stand while lined up squarely with their left side facing the camera. The patient 500 may cross their arms across their navel while interlocking their wrists. This pose may be utilized as the rest post for the thoracolumbar extension PROM test, as seen in FIG. 5A. Once again, the patient 500 may allow the clinician 501 to take them through the thoracolumbar extension range of motion, to their tolerance for the corresponding stretching procedure.

For the clinician side of the thoracolumbar extension PROM test, the clinician 501 may first ensure that the patient 500 is lined up correctly with the camera with their hands properly crossed over the naval with their wrists interlocked. The clinician 501 may then lean the patient 500 backwards while standing at the rear of the patient 500. The clinician 501 may also act as a spotter during this motion, to ensure that the patient 500 does not fall backward. The clinician 501 may also place their left hand just below the 12th rib of the patient 500 to stabilize the patient 500 while simultaneously placing their right hand on the patient's upper sternum. From here, the clinician 501 may push back on patient's sternum with their right hand while stabilizing the patient 500 with their left hand. As described above, the clinician 501 must take proper care to avoid obstructing the camera's view of the patient's knee, hip and shoulder points. Once the patient 500 has reached their point of tolerance for this PROM test, the recorded video of the patient may contain a frame of the patient 500 in this fully stretched pose, as seen in FIG. 5C. By utilizing the disclosed two dimensional pose tracking technique, the disclosed MMH system may be configured to determine the patient's thoracolumbar extension PROM angle 506, which for the patient 500 of FIG. 5D is about 22°.

In an embodiment, for a thoracolumbar extension passive range of motion measurement test of FIG. 5A-5D, a first key-point may be a point perpendicular to the floor from the femoral head of the patient's left hip, a second key-point may be the femoral head of the patient's left hip and a third key-point may be humeral head of the patient's left shoulder.

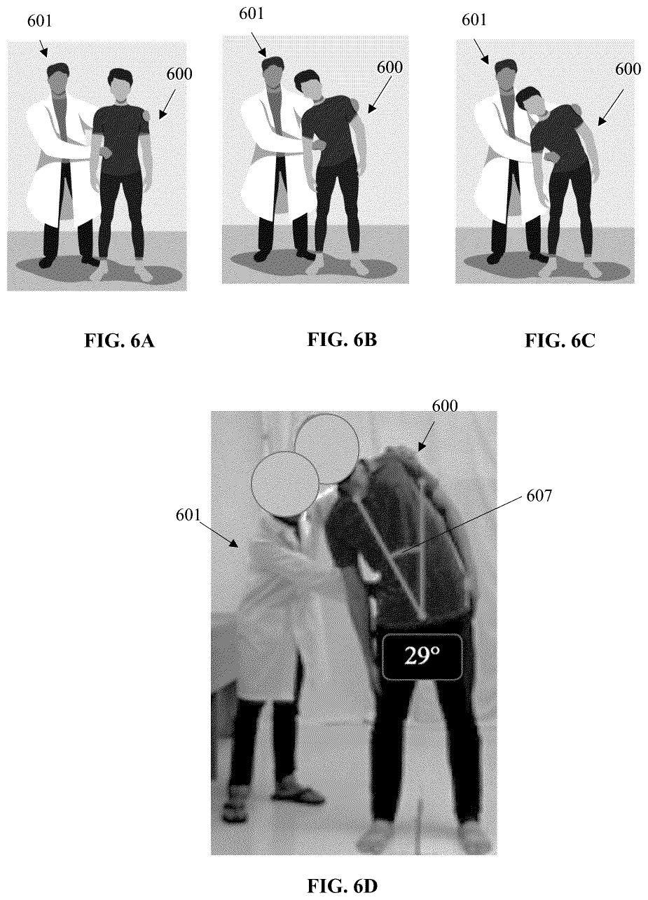

FIGS. 6A-6C illustrate the procedure for a patient 600 and clinician 601 performing a thoracolumbar right lateral flexion passive range of motion measurement test, according to an aspect. FIG. 6D illustrates an image of a patient 600 in a fully stretched pose for the thoracolumbar right lateral flexion passive range of motion test, according to an aspect.

For the patient side of the thoracolumbar right lateral flexion PROM test, the patient 600 may stand forward, facing the camera. This pose may be seen in FIG. 6A and may be utilized as the rest pose for this thoracolumbar right lateral flexion PROM test. The patient 600 may allow the clinician 601 to move them into right lateral flexion. Once achieving a fully stretched pose according to their tolerance, as seen in FIG. 6C, the patient 600 may hold the pose to allow measurements to be taken, such that the recorded video will contain frames showing the patient in this fully stretched pose.

For the clinician side of the thoracolumbar right lateral flexion PROM test, the clinician 601 may begin by making sure the patient 600 is facing square with the camera and may coach the patient 600 to minimize their thoracolumbar rotation as they bend. The clinician 601 may stand toward the right rear of the patient 600, to avoid blocking the camera's view of the patient 600. From this position, the clinician 601 may place their right hand just below the patient's 12th rib and their left hand on the patient's upper left humerus. In this position, the clinician 601 may stabilize the patient 600 with their right hand and pull with their left hand to initiate right lateral flexion for the patient 600, to the patient's tolerance. Once patient's tolerance is reached, the recorded video will contain a frame of the patient in the fully stretched pose, as seen in FIG. 6C. Again by comparing the angles between specific key-points of the patient 600 when comparing the rest pose of FIG. 6A to the fully stretched pose of FIG. 6C, the patient's thoracolumbar right lateral flexion PROM angle may be determined. In the embodiment of FIG. 6D, a patient 600 may be shown in the fully stretched pose wherein their thoracolumbar right lateral flexion PROM value 607 may be determined to be about 29°.

In an embodiment, for the thoracolumbar right lateral flexion passive range of motion measurement test of FIG. 6A-6D, a first key-point may be a point perpendicular to the midpoint between left and right anterior superior iliac spine (ASIS) of the patient's pelvis, a second key-point may be a midpoint between left and right anterior superior iliac spine (ASIS) of patient's the pelvis and a third key-point may be the suprasternal notch of the patient's sternum.

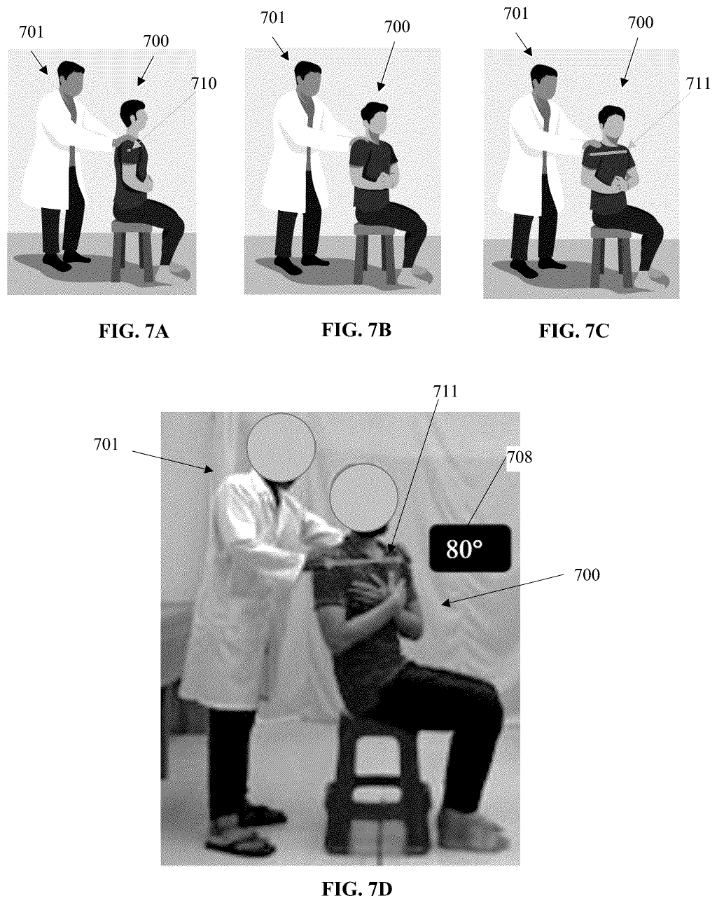

FIGS. 7A-7C illustrate the procedure for a patient 700 and clinician 701 performing a right thoracolumbar rotation passive range of motion measurement test, according to an aspect. FIG. 7D illustrates an image of a patient 700 in a fully stretched pose for the right thoracolumbar rotation passive range of motion test, according to an aspect.

For the patient side of the right thoracolumbar rotation PROM test, the patient 700 may sit on a chair height stool (the stool being approximately 18″ high, in an embodiment) with their right side squarely facing the camera. The patient 700 may also interlock their hands and wrists just above navel height and sit upright to the best of their ability while resetting their shoulders backward. The above pose may be described as the rest pose for the right thoracolumbar rotation PROM test, as seen in FIG. 7A. From this rest pose, the patient 700 may allow the clinician 701 to turn the patient's shoulders and trunk as far to the right (clockwise) as possible to the patient's tolerance, without moving the patient's hips, thus moving the patient 700 into the fully stretched pose of FIG. 7C. The clinician 701 may hold the patient 700 in this pose until a measurement is taken by the MMH system.

In an embodiment, the MMH system may be configured to identify portions of a captured video or video feed (e.g., frames of the video) wherein the patient is at rest, such as the rest pose of FIG. 7A, and the patient is fully stretched (e.g., the frame of the video where the patient has maximum movement), such as the fully stretched pose of FIG. 7C. These frames may be automatically captured by the MMH system, accordingly, wherein the MMH system may then superimpose AI-generated key-points over the corresponding frames to determine desired ROM measurements. As such, for this embodiment, the MMH system may utilize frames captured from corresponding recorded videos to measure a patient's ROM.

For the clinician side of the right thoracolumbar rotation PROM test, the clinician 701 may make sure that the patient 700 is lined up properly on the stool with their right side squarely perpendicular to the camera and centered on the camera, as well as that the patient's feet are on the floor with the patient's knees being parallel. The clinician 701 may also confirm that the patient's hands are interlocked just above their navel and their shoulders are reset backward. In order to ensure accurate results are collected, the clinician 701 may also make sure the patient 700 is sitting upright to the best of their ability. Again, this pose of the patient 700 may coincide with their rest pose, as seen in FIG. 7A. From the rest pose of FIG. 7A, The clinician 701 may place their right hand over the patient's right upper trapezius midway between the patient's neck and acromion process. Next, the clinician 701 may place their left hand on the patient's left upper back just below the spine of the scapula. The clinician 701 may then pull on the patient's right shoulder while simultaneously pushing on patient's left shoulder, applying pressure up to patient's tolerance. The clinician 701 should take care that the camera can clearly see the patient's shoulder points, while also ensuring that the patient 700 turns with minimal hip movement, to enable collection of accurate PROM results. In the embodiment of FIG. 7D showing a patient 700 in the fully stretched pose, the patient 700 may have a right thoracolumbar rotation PROM value 708 of about 80°.

In an embodiment, for the right thoracolumbar rotation passive range of motion measurement test of FIG. 7A-7D, a first key-point may be the glenohumeral joint of patients left shoulder and a second key-point may be the glenohumeral joint of the patient's right shoulder. In said embodiment, the length of the line between the glenohumeral joints of the patient may be used to determine the patient's passive range of motion angle for said PROM test. In other words, the straight line formed between the patient's glenohumeral joints while in the rest position of FIG. 7A (e.g., a resting line 710) may be compared to straight line formed between the patient's glenohumeral joints while in the fully stretched position of FIG. 7C (e.g., a stretching line 711), wherein the passive range of motion angle is measured between the resting line 710 and the stretching line 711. It is important to note that the resting line 710 may travel straight into and out of the page, such that is appears as a point in FIG. 7A.

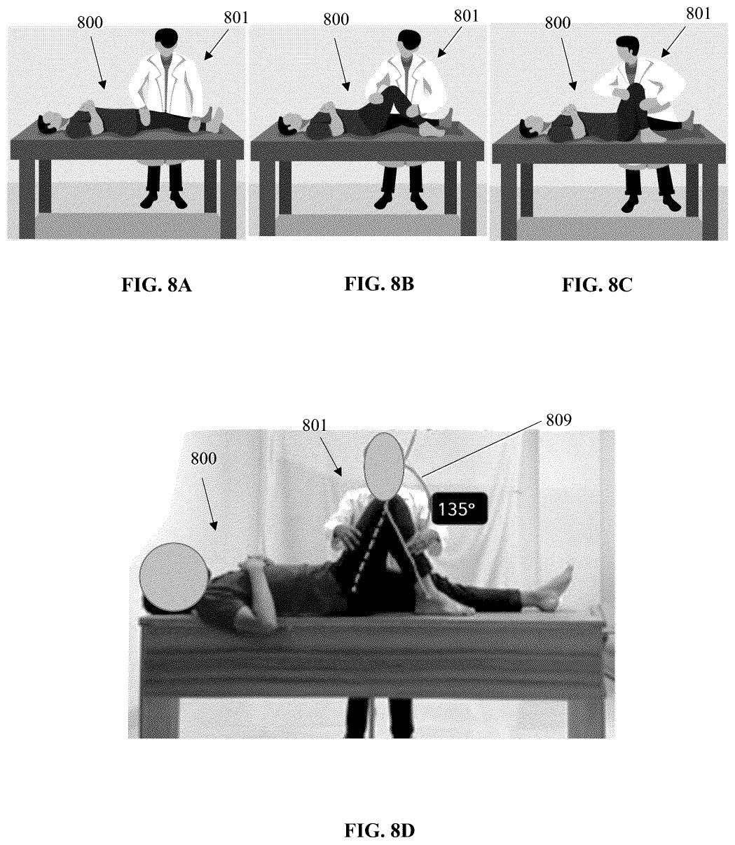

FIGS. 8A-8C illustrate the procedure for a patient 800 and clinician 801 performing a right knee flexion passive range of motion measurement test, according to an aspect. FIG. 8D illustrates an image of a patient in a fully stretched pose for the right knee flexion passive range of motion test, according to an aspect.

For the patient side of the right knee flexion PROM test, a table may be placed squarely in front of the patient 800 to lay upon. The patient 800 may lay on the table with their right side facing the camera and their right hip centered on the camera with their hands crossed over their chest. From this rest pose, as shown in FIG. 8A, the patient 800 may simply relax and allow the clinician 801 to manipulate their legs through their full range of motion, as will be described hereinbelow.

For the clinician side of the right knee flexion PROM test, the clinician 801 may stand on the side of the table, away from the camera, to avoid blocking the camera's view of the patient 800. The clinician 801 may then place their right hand on top of the right mid-thigh of the patient 800 for stabilization and their left hand two inches above the right lateral malleolus of the patient 800. From there, the clinician 801 may move the patient's right knee through the right knee flexion range of motion to the patient's tolerance. Again, this pose at which the patient 800 has been stretched to their tolerance may be described as the fully stretched pose, as seen in FIG. 8C. Next, the clinician 801 may allow the MMH system to automatically capture the PROM measurements/video while the patient is in this fully stretched pose. A patient 800 may be seen in the fully stretched pose in FIG. 8D, wherein the patient 800 has a right knee flexion PROM angle 809 of about 135°.

In an embodiment, for the right knee flexion passive range of motion measurement test of FIG. 8A-8D, a first key-point may be the femoral head of the patient's right hip, a second key-point may be the lateral femoral condyle of the patient's right leg and a third key-point may be the lateral malleolus of the patient's right ankle. As with each of the key-points listed for each of the PROM tests described above, the positions of these key-point may be compared between their corresponding rest pose, such as the rest pose of FIG. 8A, and the fully stretched pose, such as the fully stretched pose of FIG. 8C, to determine the patient's passive range of motion for a particular PROM test. In an embodiment, a line(s) may be formed between the two or more key-points, wherein the difference in angle between the line(s) in the rest pose and the fully stretched pose is utilized to determine a patient's PROM value, as described hereinabove.

It should be understood that the disclosed MMH system may utilize the video capture to identify the ideal frames/pictures (corresponding to the fully stretched pose and the rest pose) for ROM AI-based measurements. With these particular frames identified, the MMH system may save these frames/pictures, apply AI-generated key-point markers to the saved frames/pictures, and then calculate ROM measurements by comparing the key-point markers between the frames/pictures for the rest and fully stretched poses. It should also be understood that this procedure of utilizing two dimensional pose tracking to measure a patient's range of motion may not be limited solely to passive range of motion tests. In an alternative embodiment, an active range of motion test, wherein the patient 800 manipulates their own body to assume the rest pose and the fully stretched pose without the assistance of an on-site clinician 801, may utilize the same two dimensional pose tracking techniques disclosed herein.

As disclosed hereinabove, each of the described PROM tests may or may not capture and utilize an intermediate pose, depending on if said intermediate pose would prove to be useful in a determination of patient health. Other potential uses for capturing intermediate poses, such as speed and path determinations, may also provide diagnostic value when coupled with the ROM/PROM measurements. The intermediate poses disclosed herein may include FIG. 1B for left shoulder abduction, FIG. 2B for right shoulder flexion, FIG. 3B for left shoulder internal rotation, FIG. 4B for thoracolumbar flexion, FIG. 5B for thoracolumbar extension, FIG. 6B for thoracolumbar right lateral flexion, FIG. 7B for right thoracolumbar rotation and FIG. 8B for right knee flexion. Furthermore, it should be understood that moving a patient “to tolerance”, a patient being “at tolerance”, or any of their equivalents may be utilized to indicate the patient cannot stretch comfortably beyond their current position, and thus this point of tolerance may be used to define the maximal range of the patient's flexibility for a particular part(s) of their body.

While this particular pose estimation method may be described hereinabove as a health diagnostic tool, it should be understood that this pose estimation may be utilized in additional applications. For example, the disclosed pose estimation techniques, including the capture of rest, intermediate and fully stretched poses, may be utilized in sport performance diagnostic tests. Said sport performance diagnostic tests may include analyzing a golf swing or tennis stroke with an instructor in the frame, wherein path and speed of the swing are relevant. Furthermore, this same technique of tracking the movements of one party (a patient, sportsman, etc.) while ignoring movements of another party (clinician, instructor, etc.) utilizing identified key-points may be extended to a wide array of application beyond health and sports diagnostics.

It may be advantageous to set forth definitions of certain words and phrases used in this patent document. The term “couple” and its derivatives refer to any direct or indirect communication between two or more elements, whether or not those elements are in physical contact with one another. The term “or” is inclusive, meaning and/or. As used in this application, “and/or” means that the listed items are alternatives, but the alternatives also include any combination of the listed items.

The phrases “associated with” and “associated therewith,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, or the like.

Further, as used in this application, “plurality” means two or more. A “set” of items may include one or more of such items. The terms “comprising,” “including,” “carrying,” “having,” “containing,” “involving,” and the like are to be understood to be open-ended, i.e., to mean including but not limited to. Only the transitional phrases “consisting of” and “consisting essentially of,” respectively, are closed or semi-closed transitional phrases.

Throughout this description, the aspects, embodiments or examples shown should be considered as exemplars, rather than limitations on the apparatus or procedures disclosed. Although some of the examples may involve specific combinations of method acts or system elements, it should be understood that those acts and those elements may be combined in other ways to accomplish the same objectives.

Acts, elements and features discussed only in connection with one aspect, embodiment or example are not intended to be excluded from a similar role(s) in other aspects, embodiments or examples.

Aspects, embodiments or examples of the invention may be described as processes, which are usually depicted using a flowchart, a flow diagram, a structure diagram, or a block diagram. Although a flowchart may depict the operations as a sequential process, many of the operations can be performed in parallel or concurrently. In addition, the order of the operations may be re-arranged. With regard to flowcharts, it should be understood that additional and fewer steps may be taken, and the steps as shown may be combined or further refined to achieve the described methods.

Although aspects, embodiments and/or examples have been illustrated and described herein, someone of ordinary skills in the art will easily detect alternate of the same and/or equivalent variations, which may be capable of achieving the same results, and which may be substituted for the aspects, embodiments and/or examples illustrated and described herein, without departing from the scope of the invention. Therefore, the scope of this application is intended to cover such alternate aspects, embodiments and/or examples.

What is claimed is:

1. A method of measuring a patient's range of motion, operable on a computer system having a database, and a device comprising a camera, the method comprising:
recording a video of the patient as they proceed through a stretching procedure, the video comprising:
a frame of the patient in a rest pose and
a frame of the patient in a fully stretched pose;
capturing the frames of the patient in a rest pose and the fully stretched pose;
identifying key-points within the frames;
tracing the range of movement for the key-points in the frames;
calculating a range of motion angle by comparing key-point positions between the frames;
recording the range of motion angle; and
reporting the range of motion angle to the patient or a clinician.
